(19) United States
(12) Patent Application Publication
(10) Pub. No.: US 2026/0358318 A1
(43) Pub. Date: Nov. 12, 2026

(54) WATER QUALITY MEASUREMENT METHOD

(71) Applicant: MITSUBISHI HEAVY INDUSTRIES, LTD., Tokyo (JP)

(72) Inventors: Nobuyuki Ukai, Tokyo (JP); Naoki Ogawa, Tokyo (JP); Shinichi Okamoto, Tokyo (JP); Koudai Inoue, Tokyo (JP); Keiji Fujikawa, Tokyo (JP); Takashi Ike, Tokyo (JP)

(73) Assignee: MITSUBISHI HEAVY INDUSTRIES, LTD., Tokyo (JP)

(21) Appl. No.: 19/131,397

(22) PCT Filed: Sep. 7, 2023
(86) PCT No.: PCT/JP2023/032741

§ 371 Date: May 20, 2025

(30) Foreign Application Priority Data
Dec. 1, 2022 (JP) 2022-192574

(51) Int. Cl. G01N 33/18; G01N 31/16

(52) U.S. Cl. CPC G01N 33/1826; G01N 31/16

(57) ABSTRACT

A water quality measurement method for measuring acid consumption or alkali consumption of a specimen and concentration of a component contained in the specimen by titration includes: setting a target pH corresponding to the specimen; setting a pH range corresponding to a component contained in the specimen; supplying a titrant to the specimen after both setting the target pH and setting the pH range; calculating the acid consumption or alkali consumption of the specimen based on the amount of the titrant supplied to the specimen until the pH of the specimen reaches the target pH; and calculating the concentration of the component contained in the specimen based on the amount of the titrant supplied to the specimen until the pH of the specimen changes from an upper limit to a lower limit of the pH range or from the lower limit to the upper limit.

### TECHNICAL FIELD

The present disclosure relates to a water quality measurement method for measuring the acid consumption or alkali consumption of a specimen and the concentration of a component contained in the specimen by titration.

The present application claims priority based on Japanese Patent Application No. 2022-192574 filed on Dec. 1, 2022, the entire content of which is incorporated herein by reference.

### BACKGROUND ART

Patent Document 1 discloses a water quality measurement method that measures total alkalinity and total carbonic acid concentration in water, especially in seawater. In this water quality measurement method, acid titration is performed with the test water (seawater) being placed in a sealed specimen bottle until a predetermined pH is reached, and total alkalinity and total carbonic acid concentration are determined from the acid titer and pH electrode changes obtained from the results of the acid titration.

### CITATION LIST

### Patent Literature

Patent Document 1: JP2009-264913A

### SUMMARY

### Problems to be Solved

When measuring the acid consumption or alkali consumption of a specimen, a titrant may be supplied to the specimen until a corresponding pH is reached. Also, when measuring the concentration of a component contained in a specimen based on the amount of titrant supplied to the specimen, the pH range for obtaining the titrant volume varies depending on the component. However, the technique described in Patent Document 1 is unable to obtain the acid consumption or alkali consumption of a specimen until the pH corresponding to the specimen is reached. Further, if the pH range for determining the concentration is away from the pH for determining the acid consumption or alkali consumption, it may not be possible to simultaneously measure the concentration and the acid consumption or alkali consumption.

The present disclosure was made in view of the above, and an object thereof is to provide a water quality measurement method that can measure the acid consumption or alkali consumption of a specimen until pH corresponding to the specimen is reached, and that can measure the concentration of a component contained in the specimen along with the measurement of the acid consumption or alkali consumption.

### Solution to the Problems

To achieve the above object, a water quality measurement method according to the present disclosure is a water quality measurement method for measuring acid consumption or alkali consumption of a specimen and concentration of a component contained in the specimen by titration, including: a step of setting a target pH corresponding to the specimen; a step of setting a pH range corresponding to a component contained in the specimen; a step of supplying a titrant to the specimen after both the step of setting the target pH and the step of setting the pH range; a step of calculating the acid consumption or alkali consumption of the specimen on the basis of amount of the titrant supplied to the specimen until pH of the specimen reaches the target pH after starting to supply the titrant to the specimen; and a step of calculating the concentration of the component contained in the specimen on the basis of amount of the titrant supplied to the specimen until pH of the specimen changes from an upper limit to a lower limit of the pH range or from the lower limit to the upper limit after starting to supply the titrant to the specimen.

### Advantageous Effects

With the water quality measurement method of the present disclosure, it is possible to measure the acid consumption or alkali consumption of a specimen until pH corresponding to the specimen is reached, and it is possible to measure the concentration of a component contained in the specimen along with the acidity or alkalinity measurement.

### BRIEF DESCRIPTION OF DRAWINGS

FIG. 1 is a schematic configuration diagram of a waste treatment system according to some embodiments.

FIG. 2 is a flowchart of a water quality measurement method according to the first embodiment.

FIG. 3 is a graph showing a relationship between the supply amount of sodium hydroxide aqueous solution and the pH of a specimen in the first embodiment.

FIG. 4 is a flowchart of a water quality measurement method according to the third embodiment.

FIG. 5 is a flowchart of a method of setting a first calibration curve.

FIG. 6 is a graph showing a relationship between the supply amount of sodium hydroxide aqueous solution and the concentration of volatile fatty acids in the third embodiment.

FIG. 7 is a flowchart of a method of setting a second calibration curve.

FIG. 8 is a graph showing a relationship between the supply amount of sodium hydroxide aqueous solution and the concentration of ammonia nitrogen in the third embodiment.

FIG. 9 is a graph showing a relationship between the acidity constant and the temperature.

### DETAILED DESCRIPTION

Hereinafter, a water quality measurement method according to embodiments of the present disclosure will be described with reference to the drawings. The following embodiments are illustrative and not intended to limit the present disclosure, and various modifications are possible within the scope of technical ideas of the present disclosure.

The water quality measurement method of the present disclosure is to measure the acid consumption or alkali consumption of a specimen and the concentration of a component contained in the specimen by titration. In this disclosure, the acid consumption is a value calculated from the amount of acidic titrant supplied to the specimen until a predetermined pH is reached. The alkali consumption is a value calculated from the amount of alkaline titrant supplied to the specimen until a predetermined pH is reached. The present disclosure is based on the case where the contents of a methane fermenter in a waste treatment system for refining methane gas from waste are the specimen to be measured by the water quality measurement method of the present disclosure. The specimen may be any solution containing a solvent and solute and is not limited to a particular solution.

### (Waste Treatment System)

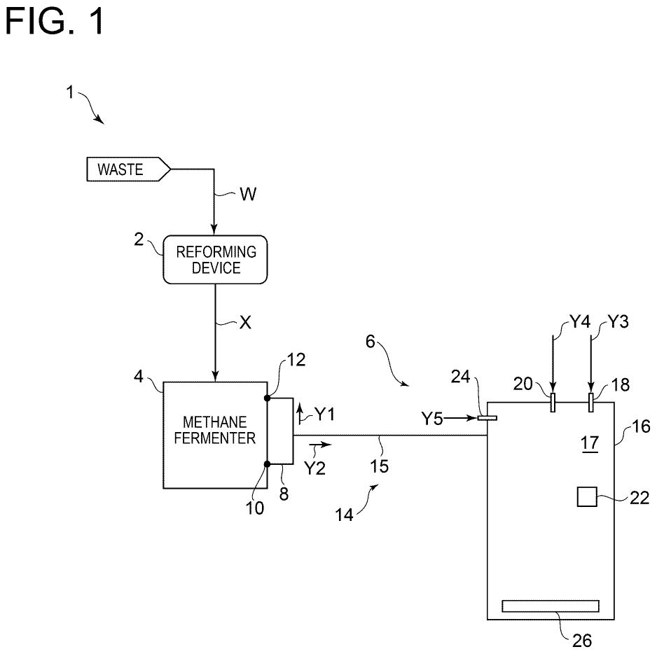

FIG. 1 is a schematic configuration diagram of a waste treatment system 1 according to some embodiments. As illustrated in FIG. 1, the waste treatment system 1 includes a reforming device 2, a methane fermenter 4, and a water quality measurement device 6.

The waste W is, for example, municipal waste. The municipal waste mainly contains kitchen waste, paper waste, and plastic waste, with a small amount of metal. The present disclosure does not limit the waste W to municipal waste. The waste W may be waste such as sludge generated by treating effluent from factories or the like and agricultural waste with a higher moisture content than municipal waste.

The reforming device 2 hydrolyzes the waste W. The reforming device 2 is, for example, a reformer configured to receive the waste W as it is from a vehicle, a plant, or the like where the waste W is collected, and hydrolyze the waste W with steam in a batch manner. The hydrolysis of the waste W in the reforming device 2 may be wet hydrolysis in which steam contacts the waste W and heats the waste W, or may be dry hydrolysis in which steam indirectly heats the waste W without contacting the waste W. Through the hydrolysis of the waste W, the waste W is broken down, and polymers contained in the waste W are converted into smaller molecules. In addition, volatile fatty acids (VFA) such as acetic acid increase. Ammonia nitrogen (NH4-N) also increases due to the decomposition of organic nitrogen contained in the waste W.

The reformed material X obtained by hydrolysis of the waste W in the reforming device 2 is supplied to the methane fermenter 4. The reformed material X contains a liquid and a solid. In an embodiment, the reformed material X is slurry with high water content, e.g., sludge.

The methane fermenter 4 microbially degrades the reformed material X supplied from the reforming device 2. More specifically, in the methane fermenter 4, the reformed material X is degraded by biological action of microorganisms, producing methane gas as a valuable resource.

In some embodiments, as illustrated in FIG. 1, the methane fermenter 4 has a circulation line 8 for taking out some of the contents of the methane fermenter 4 and returning it back into the methane fermenter 4. The circulation line 8 connects an outlet 10 formed in a lower portion of the methane fermenter 4 to an inlet 12 formed in an upper portion above the lower portion of the methane fermenter 4. The circulation line 8 takes some of the contents Y1 of the methane fermenter 4 out of the methane fermenter 4 through the outlet 10. The contents Y1 of the methane fermenter 4 flowing through the circulation line 8 is then returned to the methane fermenter 4 through the inlet 12. Thus, by circulating some of the contents Y1 of the methane fermenter 4 through the circulation line 8, the contents Y1 of the methane fermenter 4 are agitated.

The water quality measurement device 6 measures the acid consumption or alkali consumption of the contents Y1 of the methane fermenter 4 and the concentration of a component contained in the contents Y1 of the methane fermenter 4 by titration. As shown in FIG. 1, such a water quality measurement device 6 includes a specimen acquisition device 14, a reactor 16, a dropping part 18, a defoamer supply part 20, and a pH measurement device 22.

The specimen acquisition device 14 acquires the contents Y1 of the methane fermenter 4 without filtration from the methane fermenter 4. In the embodiment illustrated in FIG. 1, the specimen acquisition device 14 includes a specimen supply line 15 connecting the circulation line 8 to the reactor 16. The specimen supply line 15 branches off from the circulation line 8 so that some of the contents Y1 of the methane fermenter 4 flows from the circulation line 8 toward the reactor 16. The specimen supply line 15 is not equipped with a filter or other filtration device, and the specimen acquisition device 14 supplies some of the contents Y1 of the methane fermenter 4 to the reactor 16 as the specimen Y2 without filtration.

The reactor 16 internally has a reaction chamber 17, to which the specimen Y2 is supplied. The reactor 16 is configured such that the specimen Y2 is stored in the reaction chamber 17 in a predetermined amount. In the embodiment illustrated in FIG. 1, the water quality measurement device 6 further includes a dilution part 24 for supplying dilution water Y5 capable of diluting the specimen Y2 to the reaction chamber 17, for example, to dilute the specimen Y2 supplied from the specimen acquisition device 14 two-fold before measuring the acid consumption of the specimen Y2 or the like. The dilution part 24 is, for example, a dilution nozzle connected to a water supply. In the present disclosure, at the start of the dropwise addition, the specimen Y2 diluted two-fold is slightly alkaline, for example, with pH 8. In some embodiments, the water quality measurement device 6 measures the acid consumption of the specimen Y2 without diluting the specimen Y2 supplied from the specimen acquisition device 14. With this configuration, the use of undiluted specimen Y2 minimizes the effects of dissociation of ions of components in the specimen Y2 and changes in vapor-liquid equilibrium on measurements.

The dropping part 18 is configured to add a titrant Y3 of predetermined concentration dropwise to the reaction chamber 17. Such a dropping part 18 is, for example, a dropping nozzle attached to the upper surface of the reaction chamber 17. The titrant Y3 is, for example, sodium hydroxide aqueous solution or sulfuric acid.

The defoamer supply part 20 is configured to supply a defoamer Y4 to the reaction chamber 17. The defoamer supply part 20 is, for example, a defoamer supply nozzle attached to the upper surface of the reaction chamber 17. The defoamer Y4 may suppress the generation of foam or may break the foam that has been generated. The defoamer Y4 is, for example, an oil-based defoamer, a surfactant-based defoamer, or an alcohol-based defoamer.

The pH measurement device 22 measures the hydrogen ion concentration (pH) of the specimen Y2 in the reaction chamber 17.

In some embodiments, the water quality measurement device 6 further includes an agitation device 26 for agitating the specimen Y2 in the reaction chamber 17. The agitation device 26 includes a gas nozzle that supplies gas G to the reaction chamber 17 to agitate the specimen Y2 by aeration. In another embodiment, the agitation device 26 includes a rotor disposed in the reaction chamber 17 and rotated by magnetic force, and the specimen Y2 is agitated by the rotation of the rotor.

### First Embodiment

### (Configuration)

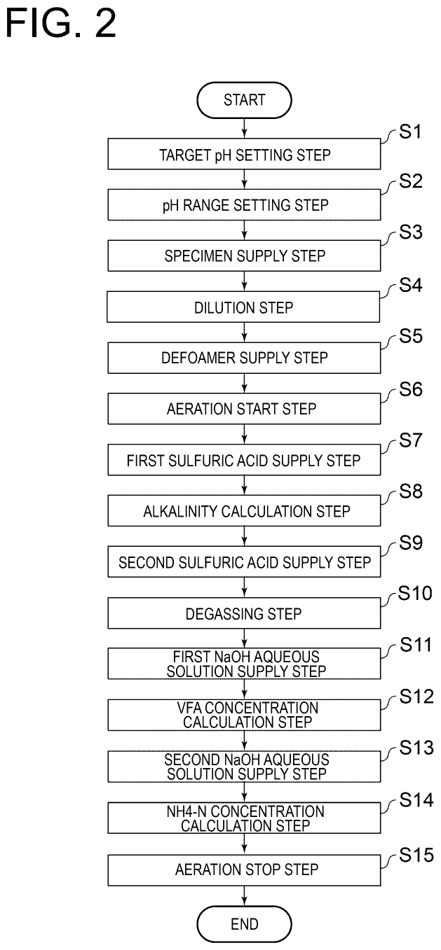

In the waste treatment system 1 illustrated in FIG. 1, the water quality measurement method for measuring the acid consumption of specimen Y2 at pH 8 and the concentration of a volatile fatty acid and ammonia nitrogen contained in the specimen Y2 by titration will be described. FIG. 2 is a flowchart of a water quality measurement method according to the first embodiment.

As illustrated in FIG. 2, the water quality measurement method includes a target pH setting step S1, a pH range setting step S2, a titrant supply step (S7, S9, S11, S13), an alkalinity calculation step S8, and a concentration calculation step (S12, S14). In the first embodiment, as illustrated in FIG. 2, the water quality measurement method further includes a specimen supply step S3, a dilution step S4, a defoamer supply step S5, an aeration start step S6, a degassing step S10, and an aeration stop step S15.

In the first embodiment, the titrant supply step includes a first sulfuric acid supply step S7, a second sulfuric acid supply step S9, a first sodium hydroxide aqueous solution supply step S11, and a second sodium hydroxide aqueous solution supply step S13. The concentration calculation step includes a volatile fatty acid concentration calculation step S12 and an ammonia nitrogen concentration calculation step S14.

In the target pH setting step S1, the target pH corresponding to the specimen Y2 is set. The target pH is a value set based on influence function I that affects the production rate of methane gas in the methane fermenter 4.

According to the inventors' findings, the rate at which methane gas is produced from acetic acid by the biological action of microorganisms is dominated by pH and is greatly affected by pH influence function I shown in Equation (1). In Equation (1), pH_{UL} is the upper limit pH at which pH inhibition does not occur, and pH_{LL} is the pH at which pH inhibition reduces the reaction rate to 5% of the maximum value. When pH_{UL} is not less than 7, the lower equation in Equation (1) is adopted, where the influence function I=1, indicating that the production rate of methane gas does not decrease. On the other hand, when pH_{UL} is less than 7, the upper equation in Equation (1) is adopted, where the biological action of microorganisms is slowed down, resulting in a rapid decrease in the production rate of methane gas. In the first embodiment, the target pH is set to pH 7.

$$I = exp ( - 3 {( \frac{pH - {pH}_{UL}}{{pH}_{UL} - {pH}_{LL}} )}^{2} ) {\vert}_{pH < {pH}_{UL}} \qquad ( 1 )$$

$$I = 1 {\vert}_{pH > {pH}_{UL}}$$

In the pH range setting step S2, the pH range corresponding to a component contained in the specimen Y2 is set. The pH range is set for each component whose concentration is to be measured. In the first embodiment, as described above, the reformed material X is produced by hydrolysis of the waste W and contains both volatile fatty acids and ammonia nitrogen. In other words, the specimen Y2 contains both volatile fatty acids and ammonia nitrogen. The pH range includes a first pH range corresponding to volatile fatty acids and a second pH range corresponding to ammonia nitrogen. The first pH range is not less than pH 2.8 and not more than pH 6.8. The second pH range is not less than pH 7.8 and not more than pH 10.8.

The first pH range will be described. The volatile fatty acids contained in the specimen Y2 are present in the form of volatile fatty acids (acetic acid, propionic acid) or volatile fatty acid ions (acetate ion, propionate ion). The acidity constants of acetic acid and propionic acid under standard conditions (the temperature of specimen Y2 is 25 °C) are both approximately 4.8. The majority of volatile fatty acids in the specimen Y2 are present in the form of acetic acid and propionic acid at pH 2.8 and below, and the majority are present in the form of acetate ion and propionate ion at pH 6.8 and above. The lower limit of the first pH range is the value at which the majority of volatile fatty acids in the specimen Y2 are present in the form of acetic acid and propionic acid. The upper limit of the first pH range is the value at which the majority of volatile fatty acids in the specimen Y2 are present in the form of acetate ion and propionate ion. The titrant Y3 (sodium hydroxide aqueous solution) added while the specimen Y2 is in the first pH range is used for the chemical reaction between volatile fatty acids and volatile fatty acid ions.

The second pH range will be described. The ammonia nitrogen contained in the specimen Y2 is present in the form of ammonia or ammonium ion. The acidity constant of ammonia nitrogen under standard conditions is approximately 9.3. The majority of ammonia nitrogen in the specimen Y2 is present in the form of ammonium ion at pH 7.8 and below, and the majority is present in the form of ammonia at pH 10.8 and above. The lower limit of the second pH range is the value at which the majority of ammonia nitrogen in the specimen Y2 is present in the form of ammonium ion. The upper limit of the second pH range is the value at which the majority of ammonia nitrogen in the specimen Y2 is present in the form of ammonia. The titrant Y3 (sodium hydroxide aqueous solution) added while the specimen Y2 is in the second pH range is used for the chemical reaction between ammonia and ammonium ion.

In the first embodiment, the target pH setting step S1 and the pH range setting step S2 are performed in this order, but the present disclosure is not limited to this embodiment. The pH range setting step S2 and the target pH setting step S1 may be performed in sequence, or the pH range setting step S2 and the target pH setting step S1 may be performed in parallel with each other.

In the specimen supply step S3, once the target pH and the pH range are set, the specimen Y2 is supplied to the reaction chamber 17. When the specimen supply step S3 is completed, the supply of the specimen Y2 to the reactor 16 is stopped, for example, by closing a valve in the specimen supply line 15. In some embodiments, the water quality measurement method further includes a draining step in which the liquid stored in the reaction chamber 17 is drained before the specimen supply step S3.

In the dilution step S4, the specimen Y2 is diluted before the titrant supply step. In the first embodiment, the dilution step S4 includes supplying dilution water Y5 to the reaction chamber 17 after the specimen supply step S3 is completed. The specimen Y2 is adjusted to pH 8, for example.

In the defoamer supply step S5, the defoamer Y4 is supplied to the reaction chamber 17 from the defoamer supply part 20 after the supply of dilution water Y5 to the reaction chamber 17 in the dilution step S4 is completed. In the aeration start step S6, the supply of gas G from the agitation device 26 (gas nozzle) to the reaction chamber 17 is started after the supply of dilution water Y5 to the reaction chamber 17 in the dilution step S4 is completed. The aeration start step S6 may be performed after the defoamer supply step S5 or in parallel with the defoamer supply step S5.

In the first sulfuric acid supply step S7 (titrant supply step), sulfuric acid (titrant Y3) is supplied to the specimen Y2 after both the target pH setting step S1 and the pH range setting step S2 are performed. In the first embodiment, the first sulfuric acid supply step S7 is performed after the aeration start step S6. Sulfuric acid is added dropwise to the specimen Y2 from the dropping part 18 until the pH of the specimen Y2 in the reaction chamber 17, as measured by the pH measurement device 22, reaches pH 7, the target pH.

In the alkalinity calculation step S8, the alkalinity (acid consumption) of the specimen Y2 is calculated on the basis of the amount of sulfuric acid supplied to the specimen Y2 until the pH of the specimen Y2 changes from pH 8 to pH 7 after starting to supply sulfuric acid to the specimen Y2 by performing the first sulfuric acid supply step S7. In some embodiments, the amount of sulfuric acid supplied to the specimen Y2 from pH 8 to pH 7 may itself be the acid consumption.

In the second sulfuric acid supply step S9 (titrant supply step), sulfuric acid is supplied to the specimen Y2 until the pH of the specimen Y2 in the reaction chamber 17, as measured by the pH measurement device 22, falls below the lower limit (less than pH 2.8) of the first pH range. In the first embodiment, sulfuric acid is added to the specimen Y2 from the dropping part 18 until pH 2 is reached.

In the degassing step S10, carbonic acid is removed from the specimen Y2 after the alkalinity calculation step S8. In the first embodiment, carbonic acid is removed from the specimen Y2 after the pH of the specimen Y2 falls below the lower limit of the first pH range by the second sulfuric acid supply step S9. Specifically, carbonic acid is removed from the specimen Y2 by the gas G supplied to the reaction chamber 17 from the agitation device 26. The concentration of carbon dioxide in the gas G is lower than the concentration of carbonic acid in the specimen Y2.

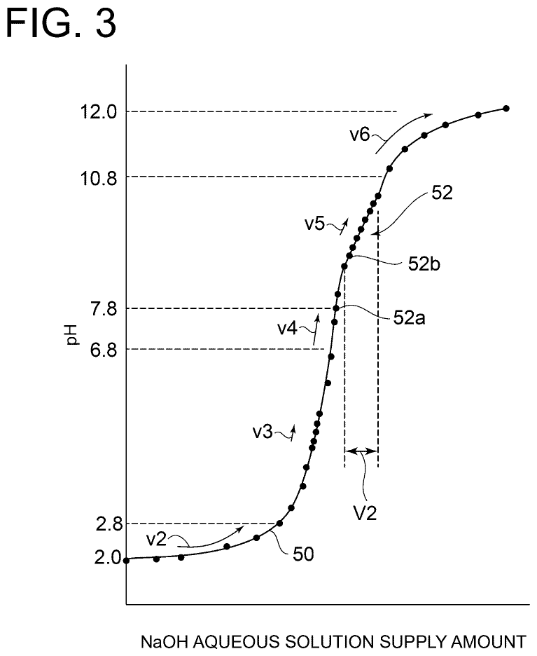

FIG. 3 is a graph showing a relationship between the supply amount of sodium hydroxide aqueous solution and the pH of the specimen Y2 in the first embodiment. In FIG. 3, the horizontal axis represents the supply amount of sodium hydroxide aqueous solution, and the vertical axis represents the pH of the specimen Y2. The graph shown in FIG. 3 has a hypothetical line 50 passing through points (pH) corresponding to the supply amount.

In the first sodium hydroxide aqueous solution supply step S11 (titrant supply step), sodium hydroxide aqueous solution (titrant Y3) is supplied to the specimen Y2 after both the target pH setting step S1 and the pH range setting step S2 are performed. In the first embodiment, the first sodium hydroxide aqueous solution supply step S11 is performed after the degassing step S10. As illustrated in FIG. 3, sodium hydroxide aqueous solution is added dropwise to the specimen Y2 from the dropping part 18 until the pH of the specimen Y2 changes from pH 2 to the upper limit (pH 6.8) of the first pH range.

In the volatile fatty acid concentration calculation step S12, the concentration of volatile fatty acids contained in the specimen Y2 is calculated on the basis of the amount of sodium hydroxide aqueous solution supplied to the specimen Y2 until the pH of the specimen Y2 changes from the lower limit to the upper limit of the first pH range (i.e., sodium hydroxide aqueous solution supplied to the specimen Y2 until the pH of the specimen Y2 changes from pH 2.8 to pH 6.8) after starting to supply the sodium hydroxide aqueous solution to the specimen Y2.

In the second sodium hydroxide aqueous solution supply step S13 (titrant supply step), sodium hydroxide aqueous solution is supplied to the specimen Y2 until the pH of the specimen Y2 in the reaction chamber 17, as measured by the pH measurement device 22, exceeds the upper limit (pH 10.8) of the second pH range. In the first embodiment, as illustrated in FIG. 3, the sodium hydroxide aqueous solution is added dropwise to the specimen Y2 from the dropping part 18 until pH 12 is reached.

In the ammonia nitrogen concentration calculation step S14, the concentration of ammonia nitrogen contained in the specimen Y2 is calculated on the basis of the amount of sodium hydroxide aqueous solution supplied to the specimen Y2 until the pH of the specimen Y2 changes from the lower limit to the upper limit of the second pH range (i.e., sodium hydroxide aqueous solution supplied to the specimen Y2 until the pH of the specimen Y2 changes from pH 7.8 to pH 10.8) after starting to supply the sodium hydroxide aqueous solution to the specimen Y2. This amount of sodium hydroxide aqueous solution is hereinafter referred to as the concentration calculation titrant volume V2.

In the aeration stop step S15, the supply of the gas G from the agitation device 26 to the reaction chamber 17 is stopped after the concentration of ammonia nitrogen contained in the specimen Y2 is calculated. In other words, the gas G is supplied to the reaction chamber 17 over a period of time from the aeration start step S6 to the aeration stop step S15. In some embodiments, the water quality measurement method further includes a washing step in which the inside of the reaction chamber 17 is washed with pure water or nitrogen-containing water, such as ammonia water, after the ammonia nitrogen concentration calculation step S14 and before the aeration stop step S15.

v1 is the supply rate of sulfuric acid in the first sulfuric acid supply step S7 (first substep). v2 is the supply rate of sodium hydroxide aqueous solution in substep S11a (second substep) of the first sodium hydroxide aqueous solution supply step S11 until the pH changes from pH 2 to pH 2.8. v3 is the supply rate of sodium hydroxide aqueous solution in substep S11b (third substep) of the first sodium hydroxide aqueous solution supply step S11 until the pH changes from pH 2.8 to pH 6.8. v4 is the supply rate of sodium hydroxide aqueous solution in substep S13a (second substep) of the second sodium hydroxide aqueous solution supply step S13 until the pH changes from pH 6.8 to pH 7.8. v5 is the supply rate of sodium hydroxide aqueous solution in substep S13b (third substep) of the second sodium hydroxide aqueous solution supply step S13 until the pH changes from pH 7.8 to pH 10.8. v6 is the supply rate of sodium hydroxide aqueous solution in substep S13c of the second sodium hydroxide aqueous solution supply step S13 until the pH changes from pH 10.8 to pH 12. In the first embodiment, v2, v4, and v6 are each higher than v1, v3, and v5.

### (Operation and Effect)

The operation and effect of the water quality measurement method according to the first embodiment will be described. As described above, in methane fermentation, when the pH of the contents Y1 of the methane fermenter 4 is less than 7, the biological action of microorganisms is slowed down, resulting in a rapid decrease in the production rate of methane gas. Therefore, general alkalinity, such as acid consumption (P alkalinity) when the contents Y1 of the methane fermenter 4 are neutralized to pH 8.3 or acid consumption (total alkalinity) when neutralized to pH 4.8, is not accurate enough to evaluate the state of the methane fermenter 4 (microbial condition).

According to the first embodiment, by setting the target pH to pH 7, it is possible to measure the acid consumption of the specimen Y2 until pH 7 is reached. Thus, the accuracy of evaluating the state of the methane fermenter 4 can be improved. Further, by setting the first and second pH ranges, the concentrations of volatile fatty acids and ammonia nitrogen in the specimen Y2 can also be obtained. Thus, the accuracy of evaluating the state of the methane fermenter 4 can be further improved.

In some embodiments, the first pH range is not less than pH 4.3 and not more than pH 5.3. As described above, the acidity constants of acetic acid and propionic acid under standard conditions are both approximately 4.8. Therefore, when the pH is between pH 4.3 and 5.3, the effect of ions other than acetate ion and propionate ion are suppressed (in other words, changes in pH of the specimen Y2 are dominated by acetate ion and propionate ion). Therefore, by setting the first pH range to between pH 4.3 and 5.3, the measurement accuracy of concentration of volatile fatty acids in the specimen Y2 can be improved.

In some embodiments, the second pH range is not less than pH 8.8 and not more than pH 9.8. As described above, the acidity constant of ammonia nitrogen under standard conditions is approximately 9.3. Therefore, when the pH is between pH 8.8 and 9.8, the effect of ions other than ammonia and ammonium ion are suppressed (in other words, changes in pH of the specimen Y2 are dominated by ammonia and ammonium ion). Therefore, by setting the second pH range to between pH 8.8 and 9.8, the accuracy of calculating the concentration of ammonia nitrogen in the specimen Y2 can be improved.

If the specimen Y2 contains carbonic acid, the measurement accuracy of concentrations of volatile fatty acids and ammonia nitrogen in the specimen Y2 may decrease. More specifically, carbonic acid in the specimen Y2 is present in one of the following forms: carbonic acid, bicarbonate ion, or carbonate ion. The acidity constant at the first stage (acidity constant between carbonic acid and bicarbonate ion) under standard conditions is approximately 6.3. Therefore, the sodium hydroxide aqueous solution is used for the chemical reaction between carbonic acid and bicarbonate ion in the supply (dropwise addition) of sodium hydroxide aqueous solution from the lower limit (pH 2.8) to the upper limit (pH 6.8) of the first pH range in the first sodium hydroxide aqueous solution supply step S11. As a result, the concentration of volatile fatty acids in the specimen Y2 may be measured to be higher than it actually is. Similarly, the acidity constant at the second stage (acidity constant between carbonic acid and bicarbonate ion) under standard conditions is approximately 10.3. Therefore, the sodium hydroxide aqueous solution is used for the chemical reaction between bicarbonate ion and carbonate ion in the supply (dropwise addition) of sodium hydroxide aqueous solution from the lower limit (pH 7.8) to the upper limit (pH 10.8) of the second pH range in the second sodium hydroxide aqueous solution supply step S13. As a result, the concentration of ammonia nitrogen in the specimen Y2 may be measured to be higher than it actually is.

In the first embodiment, the water quality measurement device 6 is used to measure the acid consumption or the like of the specimen Y2 at a temperature of 25 °C, but the temperature of the specimen Y2 is not limited to 25 °C. In some embodiments, the water quality measurement device 6 is configured to maintain the temperature of the specimen Y2 at a predetermined temperature, and each of the first pH range and the second pH range is corrected based on the predetermined temperature.

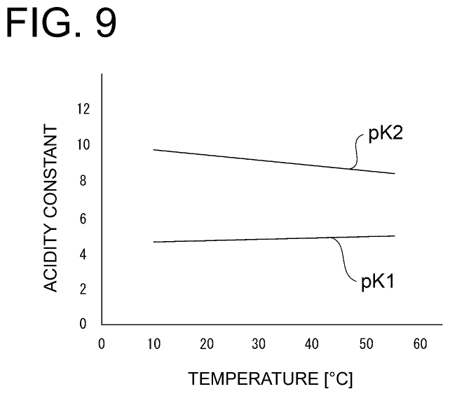

An example of correction of the first pH range and the second pH range according to some embodiments will be described. FIG. 9 is a graph showing a relationship between the acidity constant and the temperature. FIG. 9 shows the acidity constant pK1 for acetic acid (pK1=−log K1) and acidity constant pK2 for ammonia nitrogen (pK2=−log K2). As shown in FIG. 9, the acidity constant changes depending on the temperature. The acidity constant pK1 of acetic acid increases slightly with an increase in temperature. The acidity constant pK2 of ammonia nitrogen decreases with an increase in temperature.

In the first embodiment described above, the first pH range is defined by using the acidity constant of acetic acid in the standard condition (approximately 4.8) as the median, with the upper and lower limits determined by adding and subtracting a predetermined value (2.0) to/from the median. As shown in FIG. 9, the acidity constant of acetic acid is determined by the temperature. If the temperature of the specimen Y2 is 55 °C, the acidity constant of acetic acid is 4.85, and the first pH range is corrected to between pH 2.85 and 6.85, which is determined by adding and subtracting a predetermined value (2.0) to/from 4.85. The predetermined value in the first pH range is constant regardless of temperature. With this correction, the first pH range is corrected based on the temperature of the specimen Y2, and the temperature dependence of acetic acid is taken into account. Thus, the accuracy of calculating the concentration of acetic acid in the specimen Y2 can be improved. The case in which the volatile fatty acid is acetic acid has been described as an example, but the volatile fatty acid may be propionic acid instead of acetic acid or may contain both acetic acid and propionic acid.

In the first embodiment described above, the second pH range is defined by using the acidity constant of ammonia nitrogen in the standard condition (approximately 9.3) as the median, with the upper and lower limits determined by adding and subtracting a predetermined value (1.5) to/from the median. As shown in FIG. 9, the acidity constant of ammonia nitrogen is determined by the temperature. If the temperature of the specimen Y2 is 55 °C, the acidity constant of ammonia nitrogen is 8.45, and the second pH range is between pH 6.95 and 9.95, which is determined by adding and subtracting a predetermined value (1.5) to/from 8.45. The predetermined value in the second pH range is constant regardless of temperature. The predetermined value in the first pH range and the predetermined value in the second pH range may be the same or different from each other. With this correction, the second pH range is corrected based on the temperature of the specimen Y2, and the temperature dependence of ammonia nitrogen is taken into account. Thus, the accuracy of calculating the concentration of ammonia nitrogen in the specimen Y2 can be improved.

According to the first embodiment, the concentration of volatile fatty acids in the specimen Y2 is calculated on the basis of the amount of sodium hydroxide aqueous solution supplied until the pH of the specimen Y2 changes from the lower limit to the upper limit of the first pH range after removing carbonic acid from the specimen Y2 by performing the degassing step S10. Similarly, the concentration of ammonia nitrogen in the specimen Y2 is calculated on the basis of the amount of sodium hydroxide aqueous solution supplied until the pH of the specimen Y2 changes from the lower limit to the upper limit of the second pH range after removing carbonic acid from the specimen Y2 by performing the degassing step S10. This suppresses a decrease in the measurement accuracy of concentration of volatile fatty acids and ammonia nitrogen due to the presence of carbonic acid in the specimen Y2.

In the first sodium hydroxide aqueous solution supply step S11, substep S11a corresponds to the preparatory stage for performing substep S11b. Therefore, during the substep S11a, the sodium hydroxide aqueous solution does not need to be supplied as carefully as in the first sulfuric acid supply step S7 and the substep S11b. Similarly, in the second sodium hydroxide aqueous solution supply step S13, substep S13a corresponds to the preparatory step for performing substep S13b. Therefore, during the substep S13b, the sodium hydroxide aqueous solution does not need to be supplied as carefully as in the first sulfuric acid supply step S7 and the substep S13b. According to the first embodiment, by making v2 and v4 higher than v1, v3 and v5, the time to measure the alkalinity of the specimen Y2 and the concentration of volatile fatty acids and the concentration of ammonia nitrogen in the specimen Y2 by titration can be reduced.

In the first embodiment, the case in which the specimen Y2 is the contents of the methane fermenter 4 has been described as an example, but the present disclosure is not limited to this embodiment. For example, the specimen Y2 may be the contents of a saccharification tank for producing sugar as valuables from carbohydrates such as starch and cellulose, or the contents of a composting device for producing compost by composting carbohydrates. In some embodiments, the specimen Y2 is the contents of a fermenter for microbially degrading a reformed material X obtained by hydrolysis of waste W.

Although the water quality measurement method according to the first embodiment is used to measure the acid consumption of the specimen Y2, the present disclosure is not limited to this embodiment. In some embodiments, the water quality measurement method according to the present disclosure measures the alkali consumption of the specimen Y2 and the concentration of a component contained in the specimen Y2 by titration. The water quality measurement method according to the present disclosure does not limit the target pH to pH 7, but the target pH can be freely set according to the specimen Y2. Similarly, the water quality measurement method of the present disclosure does not limit the pH range to the first pH range (pH 2.8 to pH 6.8) or the second pH range (pH 7.8 to pH 10.8), but the pH range can be freely set according to the component whose concentration is to be measured.

### Second Embodiment

The water quality measurement method according to the second embodiment will be described. In the second embodiment, the method of calculating the concentration of ammonia nitrogen differs from the first embodiment. The configuration is otherwise the same as that described in the first embodiment. In the second embodiment, the same constituent elements as those in the first embodiment are associated with the same reference characters and thus not described again in detail.

In the second embodiment, in the ammonia nitrogen concentration calculation step S14, the concentration calculation titrant volume V2 is calculated on the basis of the pH change in the second pH range of the specimen Y2 caused by the supply of sodium hydroxide aqueous solution (titrant Y3).

As illustrated in FIG. 3, the portion 52 of the line 50 from pH 7.8 to pH 10.8 can be considered to extend in a straight line. Therefore, by obtaining the slope of the portion 52, the concentration calculation titrant volume V2 can be calculated. In the second embodiment, the slope of the portion 52 is calculated from two points 52a and 52b that are at different position from each other in the portion 52. The point 52a is pH 7.8, and the point 52b is values other than pH 7.8 and pH 10.8.

The operation and effect of the water quality measurement method according to the second embodiment will be described. According to the second embodiment, the concentration calculation titrant volume V2 is calculated before the sodium hydroxide aqueous solution is actually supplied up to the concentration calculation titrant volume V2, and the concentration of ammonia nitrogen in the specimen Y2 is calculated on the basis of the calculated concentration calculation titrant volume V2. This shortens the time required to measure the concentration of ammonia nitrogen.

In some embodiments, in the volatile fatty acid concentration calculation step S12, the amount of sodium hydroxide aqueous solution supplied to the specimen Y2 until the pH of the specimen Y2 changes from pH 2.8 to pH 6.8 is calculated on the basis of the pH change in the first pH range of the specimen Y2 caused by the supply of sodium hydroxide aqueous solution (titrant Y3).

### Third Embodiment

The water quality measurement method according to the third embodiment will be described. The water quality measurement method according to the third embodiment is the water quality measurement method according to the first embodiment with the addition of a calibration curve setting step S16. In the third embodiment, the same constituent elements as those in the first embodiment are associated with the same reference characters and thus not described again in detail. The water quality measurement method according to some embodiments is the water quality measurement method according to the second embodiment with the addition of a calibration curve setting step S16.

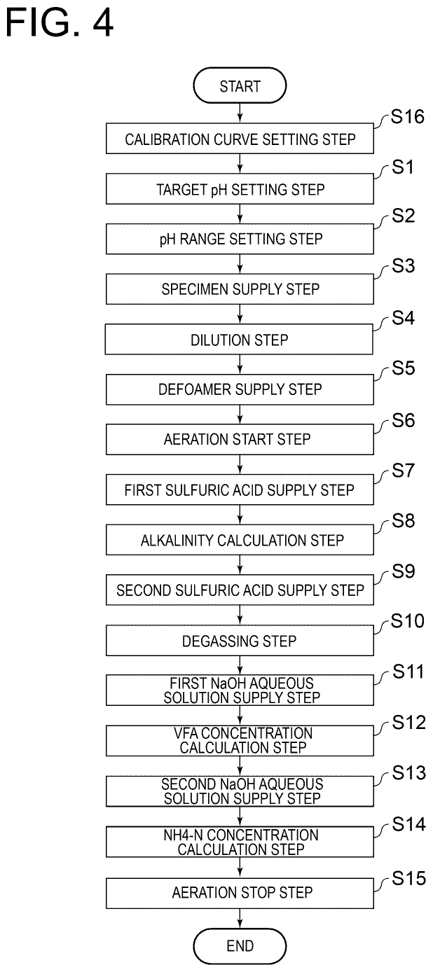

FIG. 4 is a flowchart of the water quality measurement method according to the third embodiment. As illustrated in FIG. 4, the water quality measurement method further includes a calibration curve setting step S16 performed before the first sulfuric acid supply step S7. In the third embodiment, the calibration curve setting step S16 is performed before the target pH setting step S1.

In the calibration curve setting step S16, a calibration curve is set to show the relationship between the concentration of a component in the specimen Y2 and the supply amount of titrant Y3. In the third embodiment, a first calibration curve L1 is set to show the relationship between the concentration of volatile fatty acids in the specimen Y2 and the supply amount of sodium hydroxide aqueous solution. Further, a second calibration curve L2 is set to show the relationship between the concentration of ammonia nitrogen in the specimen Y2 and the supply amount of sodium hydroxide aqueous solution. That is, in the calibration curve setting step S16, both the first calibration curve L1 and the second calibration curve L2 are set. In some embodiments, in the calibration curve setting step S16, one of the first calibration curve L1 or the second calibration curve L2 is set.

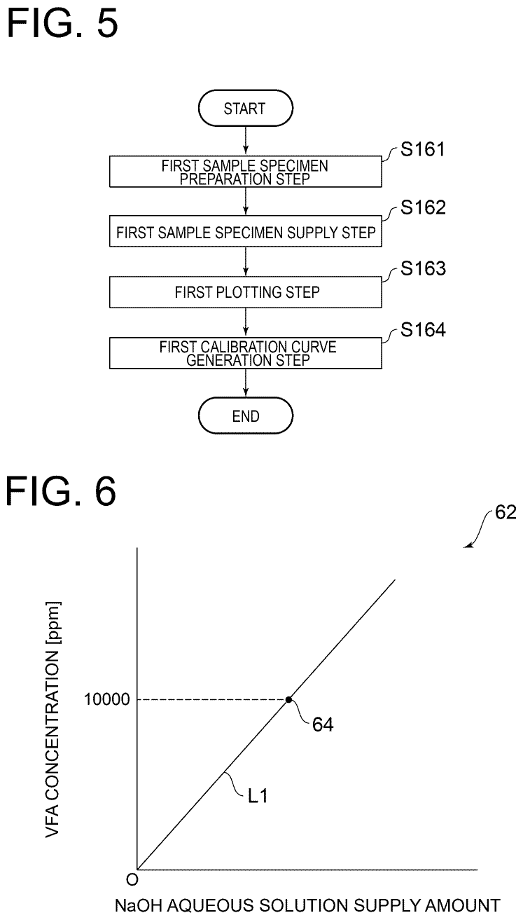

An example of the method of setting the first calibration curve L1 will be described. FIG. 5 is a flowchart of the method of setting the first calibration curve L1. As illustrated in FIG. 5, the method of setting the first calibration curve L1 includes a first sample specimen preparation step S161, a first sample specimen supply step S162, a first plotting step S163, and a first calibration curve generation step S164.

In the first sample specimen preparation step S161, a first sample specimen 60 of known concentration containing volatile fatty acids within a concentration range of not less than 9500 ppm and not more than 10500 ppm is prepared. For example, the concentration of volatile fatty acids in the first sample specimen 60 is 10000 ppm.

In the first sample specimen supply step S162, sodium hydroxide aqueous solution (titrant Y3) is supplied to the first sample specimen 60. This sodium hydroxide aqueous solution is the same as the sodium hydroxide aqueous solution supplied to the specimen Y2 in the first sodium hydroxide aqueous solution supply step S11 and the second sodium hydroxide aqueous solution supply step S13.

FIG. 6 is a graph showing a relationship between the supply amount of sodium hydroxide aqueous solution and the concentration of volatile fatty acids in the third embodiment. The graph shown in FIG. 6 includes a first coordinate system 62 with the horizontal axis representing the supply amount of sodium hydroxide aqueous solution and the vertical axis representing the concentration of volatile fatty acids.

In the first plotting step S163, a first point 64 corresponding to the concentration of volatile fatty acids (10000 ppm) in the first sample specimen 60 is plotted on the first coordinate system 62. In the first calibration curve generation step S164, a straight line passing through the first point 64 and the origin O of the first coordinate system 62 is generated as a calibration curve (first calibration curve L1) for volatile fatty acids.

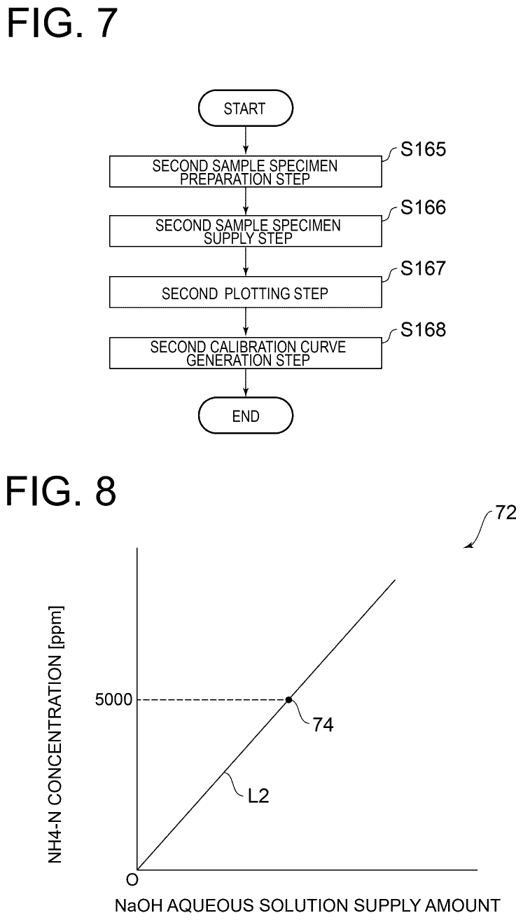

An example of the method of setting the second calibration curve L2 will be described. FIG. 7 is a flowchart of the method of setting the second calibration curve L2. As illustrated in FIG. 7, the method of setting the second calibration curve L2 includes a second sample specimen preparation step S165, a second sample specimen supply step S166, a second plotting step S167, and a second calibration curve generation step S168.

In the second sample specimen preparation step S165, a second sample specimen 70 of known concentration containing ammonia nitrogen within a concentration range of not less than 4500 ppm and not more than 5500 ppm is prepared. For example, the concentration of ammonia nitrogen in the second sample specimen 70 is 5000 ppm.

In the second sample specimen supply step S166, sodium hydroxide aqueous solution (titrant Y3) is supplied to the second sample specimen 70. This sodium hydroxide aqueous solution is the same as the sodium hydroxide aqueous solution supplied to the specimen Y2 in the first sodium hydroxide aqueous solution supply step S11 and the second sodium hydroxide aqueous solution supply step S13.

FIG. 8 is a graph showing a relationship between the supply amount of sodium hydroxide aqueous solution and the concentration of ammonia nitrogen in the third embodiment. The graph shown in FIG. 8 includes a second coordinate system 72 with the horizontal axis representing the supply amount of sodium hydroxide aqueous solution and the vertical axis representing the concentration of ammonia nitrogen.

In the second plotting step S167, a second point 74 corresponding to the concentration of ammonia nitrogen (5000 ppm) in the second sample specimen 70 is plotted on the second coordinate system 72. Then, in the second calibration curve generation step S168, a straight line passing through the second point 74 and the origin O of the second coordinate system 72 is generated as a calibration curve (second calibration curve L2) for ammonia nitrogen.

In the volatile fatty acid concentration calculation step S12, the concentration of volatile fatty acids is calculated on the basis of the first calibration curve L1 and the amount of sodium hydroxide aqueous solution supplied to the specimen Y2 until the pH of the specimen Y2 changes from pH 2.8 to pH 6.8. Specifically, the concentration of volatile fatty acids corresponding to the amount of sodium hydroxide aqueous solution supplied to the specimen Y2 is obtained from the first calibration curve L1.

In the ammonia nitrogen concentration calculation step S14, the concentration of ammonia nitrogen is calculated on the basis of the concentration calculation titrant volume V2 and the second calibration curve L2. Specifically, the concentration of ammonia nitrogen corresponding to the concentration calculation titrant volume V2 is obtained from the second calibration curve L2.

The operation and effect of the water quality measurement method according to the third embodiment will be described. According to the third embodiment, compared to the case where the first calibration curve L1 and the second calibration curve L2 are not used, the measurement accuracy of concentration of volatile fatty acids in the specimen Y2 and the measurement accuracy of concentration of ammonia nitrogen can be improved.

When measuring the concentration of volatile fatty acids, it may be desirable to have a high measurement accuracy of concentration around 10000 ppm. This is because when the concentration of volatile fatty acids exceeds 10000 ppm, the microbial degradation of the reformed material X is inhibited. According to the third embodiment, since the calibration curve (first calibration curve L1) for volatile fatty acids is a straight line passing through the first point 64 of the first sample specimen 60 with a concentration of volatile fatty acids of 10000 ppm and the origin on the first coordinate system 62, the measurement accuracy of concentration of volatile fatty acids around 10000 ppm can be improved.

The present disclosure is not limited to the case where the concentration of volatile fatty acids in the first sample specimen 60 is in the range of not less than 9500 ppm and not more than 10500 ppm. In some embodiments, in the first sample specimen preparation step S161, a first sample specimen 60 containing volatile fatty acids within a concentration range of not less than 19000 ppm and not more than 21000 ppm is prepared.

When measuring the concentration of ammonia nitrogen, it may be desirable to have a high measurement accuracy of concentration around 5000 ppm. This is because when the concentration of ammonia nitrogen exceeds 5000 ppm, the microbial degradation of the reformed material X is inhibited. According to the third embodiment, since the calibration curve (second calibration curve L2) for ammonia nitrogen is a straight line passing through the second point 74 of the second sample specimen 70 with a concentration of ammonia nitrogen of 5000 ppm and the origin on the second coordinate system 72, the measurement accuracy of concentration of ammonia nitrogen around 5000 ppm can be improved.

The present disclosure is not limited to the case where the concentration of ammonia nitrogen in the second sample specimen 70 is in the range of not less than 9500 ppm and not more than 10500 ppm.

In some embodiments, in the second sample specimen preparation step S165, a second sample specimen 70 containing ammonia nitrogen within a concentration range of not less than 3800 ppm and not more than 4200 ppm is prepared. The methane fermenter 4 may ferment the reformed material X using microorganisms under medium temperature (37 °C±5 °C) conditions. In this case, it may be desirable to have a high measurement accuracy of concentration of ammonia nitrogen around 4000 ppm. This method can improve the accuracy of measuring the concentration of ammonia nitrogen around 4000 ppm when the methane fermenter 4 is in medium-temperature fermentation.

In some embodiments, in the second sample specimen preparation step S165, a second sample specimen 70 containing ammonia nitrogen within a concentration range of not less than 2850 ppm and not more than 3150 ppm is prepared. The methane fermenter 4 may ferment the reformed material X using microorganisms under high temperature (55 °C±5 °C) conditions. In this case, it may be desirable to have a high measurement accuracy of concentration of ammonia nitrogen around 3000 ppm. This method can improve the accuracy of measuring the concentration of ammonia nitrogen around 4000 ppm when the methane fermenter 4 is in high-temperature fermentation.

In the third embodiment, the calibration curve is a straight line, but the present disclosure is not limited to this embodiment. The calibration curve may be, for example, a curve. In the third embodiment, one point is plotted on the coordinate system, but multiple points may be plotted on the coordinate system.

The method of setting the calibration curve is not limited, and can be, for example, an absolute calibration curve method or a standard addition method. The absolute calibration curve method is a method of obtaining a calibration curve by preparing a sample specimen of known concentration and measuring a unique signal of the water quality measurement device 6 to calculate the relationship between the concentration and the signal. The standard addition method is a method of obtaining a calibration curve by preparing a sample specimen of known concentration by directly adding at least one of volatile fatty acid and ammonia nitrogen to the specimen Y2, whose water quality is to be measured, and measuring a unique signal of the water quality measurement device 6 to calculate the relationship between the concentration and the signal.

In the third embodiment, the measurement accuracy of concentration of components in the specimen is improved by setting the calibration curve, but the measurement accuracy may be improved by other methods. For example, the water quality measurement method may further include a step of setting a correction factor K calculated by Z1/Z2, with 10000 ppm as the set value Z1 and the amount of titrant Y3 supplied to the specimen Y2 from the lower limit to the upper limit of the pH range as the set value Z2. In the concentration calculation step (S12, S14), the concentration of the component in the specimen Y2 is calculated on the basis of the amount of titrant Y3 supplied to the specimen Y2 from the lower limit to the upper limit of the pH range, multiplied by the correction factor K.

The contents described in the above embodiments would be understood as follows, for instance.

[1] A water quality measurement method according to the present disclosure is a water quality measurement method for measuring acid consumption or alkali consumption of a specimen (Y2) and concentration of a component contained in the specimen by titration, including: a step (S1) of setting a target pH corresponding to the specimen; a step (S2) of setting a pH range corresponding to a component contained in the specimen; a step (S7, S9, S11, S13) of supplying a titrant (Y3) to the specimen after both the step of setting the target pH and the step of setting the pH range; a step (S8) of calculating the acid consumption or alkali consumption of the specimen on the basis of amount of the titrant supplied to the specimen until pH of the specimen reaches the target pH after starting to supply the titrant to the specimen; and a step (S12, S14) of calculating the concentration of the component contained in the specimen on the basis of amount of the titrant supplied to the specimen until pH of the specimen changes from an upper limit to a lower limit of the pH range or from the lower limit to the upper limit after starting to supply the titrant to the specimen.

With the above method [1], by setting the target pH corresponding to the specimen, it is possible to measure the acid consumption or alkali consumption of the specimen until pH corresponding to the specimen is reached. Additionally, with the above method [1], by setting the pH range corresponding to a component contained in the specimen, the concentration of the component in the specimen can also be obtained.

[2] In some embodiments, in the above method [1], the specimen is contents of a fermenter for microbially degrading a reformed material (X) obtained by hydrolysis of waste (W).

With the above configuration [2], it is possible to measure the acid consumption or alkali consumption of the contents of the fermenter until pH corresponding to the contents of the fermenter is reached as well as the concentration of a component in the contents of the fermenter.

[3] In some embodiments, in the above method [2], the fermenter is a methane fermenter (4) which produces methane gas by microbially degrading the reformed material, and the step of setting the target pH includes setting the target pH on the basis of an influence function (I) that affects a production rate of the methane gas in the methane fermenter.

With the above method [3], since the target pH is set on the basis of the influence function, it is possible to measure the acid consumption or alkali consumption of the contents of the methane fermenter, taking into account the production rate of methane gas. Thus, the accuracy of evaluating the state of the methane fermenter can be improved compared to measuring general alkalinity (e.g., P alkalinity or total alkalinity).

[4] In some embodiments, in the above method [3], the target pH is pH 7, and the step of calculating the acid consumption or alkali consumption of the specimen includes calculating the alkalinity of the specimen on the basis of amount of the titrant supplied to the specimen until pH of the specimen reaches pH 7 after starting to supply the titrant to the specimen.

According to the inventors' findings, the acid consumption until pH 7 is reached is the value at which the state of the methane fermenter can be evaluated with high accuracy. With the above method [4], the acid consumption until the pH of the contents of the methane fermenter reaches pH 7 is calculated, which enables highly accurate evaluation of the state of the methane fermenter.

[5] In some embodiments, in any one of the above methods [2] to [4], the component contained in the specimen includes a volatile fatty acid, and the pH range includes a first pH range of not less than pH 2.8 and not more than pH 6.8.

With the above method [5], it is possible to measure the concentration of volatile fatty acids in the contents of the fermenter.

[6] In some embodiments, in the above method [5], the first pH range is not less than pH 4.3 and not more than pH 5.3.

With the above method [6], it is possible to improve the measurement accuracy of concentration of volatile fatty acids in the contents of the fermenter.

[7] In some embodiments, in the above method [5], the first pH range is corrected on the basis of temperature of the specimen.

The inventors have found that the accuracy of calculating the concentration of volatile fatty acids in the contents of the fermenter can be improved by correcting the pH range to an appropriate range depending on the temperature of the specimen. With the above method [7], since the pH range is corrected on the basis of temperature of the specimen, it is possible to improve the measurement accuracy of concentration of volatile fatty acids in the contents of the fermenter.

[8] In some embodiments, in any one of the above methods [2] to [7], the component contained in the specimen includes ammonia nitrogen, and the pH range includes a second pH range of not less than pH 7.8 and not more than pH 10.8.

With the above method [8], it is possible to measure the concentration of ammonia nitrogen in the contents of the fermenter.

[9] In some embodiments, in the above method [8], the second pH range is not less than pH 8.8 and not more than pH 9.8.

With the above method [9], it is possible to improve the measurement accuracy of concentration of ammonia nitrogen in the contents of the fermenter.

[10] In some embodiments, in the above method [8], the second pH range is corrected on the basis of temperature of the specimen.

The inventors have found that the accuracy of calculating the concentration of ammonia nitrogen in the contents of the fermenter can be improved by correcting the pH range to an appropriate range depending on the temperature of the specimen. With the above method [10], since the pH range is corrected on the basis of temperature of the specimen, it is possible to improve the measurement accuracy of concentration of ammonia nitrogen in the contents of the fermenter.

[11] In some embodiments, in any one of the above methods [1] to [10], the method further includes a step (S10) of removing carbonic acid from the specimen after calculating the acid consumption of the specimen in the step of calculating the acid consumption or alkali consumption of the specimen. The step of calculating the concentration of the component contained in the specimen includes calculating the concentration of the component contained in the specimen on the basis of amount of the titrant supplied to the specimen until pH of the specimen after removing carbonic acid changes from the lower limit to the upper limit of the pH range.

If the specimen contains carbonic acid, the measurement accuracy of the component in the specimen may decrease. With the above method [11], the concentration of the component in the specimen is calculated on the basis of the amount of titrant until the pH of the specimen after removing carbonic acid changes from the lower limit to the upper limit of the pH range. This suppresses a decrease in the measurement accuracy of concentration due to the presence of carbonic acid in the specimen.

[12] In some embodiments, in any one of the above methods [1] to [11], the step of supplying the titrant to the specimen includes: a first substep (S7) of supplying the titrant to the specimen at a first supply rate (v1) until pH of the specimen reaches the target pH after starting to supply the titrant to the specimen; a second substep (S11a, S13a) of supplying the titrant to the specimen at a second supply rate (v2, v4) until pH of the specimen reaches the pH range after the first substep; and a third substep (S11b, S13b) of supplying the titrant to the specimen at a third supply rate (v3, v5) until pH of the specimen reaches the upper limit or the lower limit of the pH range after the second substep. The second supply rate is higher than each of the first supply rate and the third supply rate.

The second substep corresponds to the preparatory stage for performing the third substep, and the titrant does not need to be supplied as carefully as in the first and third substeps. With the above method [12], since the second supply rate is higher than each of the first supply rate and the third supply rate, the time required to measure the acid consumption or alkali consumption of the specimen and the concentration of the component in the specimen by titration can be reduced.

[13] In some embodiments, in any one of the above methods [1] to [12], the step of calculating the concentration of the component contained in the specimen includes calculating a concentration calculation titrant volume (V2) on the basis of a pH change within the pH range of the specimen caused by the supply of the titrant, where the concentration calculation titrant volume is amount of the titrant supplied to the specimen until pH of the specimen changes from the upper limit to the lower limit of the pH range or from the lower limit to the upper limit.

With the above method [13], the concentration calculation titrant volume is calculated before the titrant is actually supplied up to the concentration calculation titrant volume, and the time required to measure the concentration of the component in the specimen can be reduced.

[14] In some embodiments, in any one of the above methods [1] to [13], the method further includes a step (S4) of diluting the specimen prior to the step of supplying the titrant to the specimen.

With the above method [14], it is possible to reduce the viscosity of the specimen and facilitate agitation of the specimen. This accelerates the reaction between the specimen and the titrant.

[15] In some embodiments, in any one of the above methods [1] to [14], the method further includes a step (S16) of setting a calibration curve indicating a relationship between concentration of the component contained in the specimen and supply amount of the titrant prior to the step of supplying the titrant to the specimen. The step of calculating the concentration of the component contained in the specimen includes calculating the concentration of the component contained in the specimen on the basis of the calibration curve (L1, L2) and amount of the titrant supplied to the specimen until pH of the specimen changes from the upper limit to the lower limit of the pH range or from the lower limit to the upper limit after starting to supply the titrant to the specimen.

With the above method [15], compared to the case where the calibration curve is not used, the measurement accuracy of concentration of the component in the specimen can be improved.

[16] In some embodiments, in the above method [15], the component contained in the specimen includes a volatile fatty acid. The step of setting the calibration curve includes: a step (S161) of preparing a first sample specimen (60) containing the volatile fatty acid of known concentration; a step (S162) of supplying the titrant to the first sample specimen; a step (S163) of plotting a first point (64) corresponding to concentration of the volatile fatty acid contained in the first sample specimen on a first coordinate system (62) with a horizontal axis representing the amount of the titrant and a vertical axis representing the concentration of the volatile fatty acid; and a step (S164) of defining a straight line that passes through the first point and an origin of the first coordinate system as the calibration curve (L1) of the volatile fatty acid.

With the above method [16], it is possible to generate a calibration curve that improves the accuracy of measuring the concentration of volatile fatty acids in the specimen.

[17] In some embodiments, in the above method [16], the first sample specimen contains the volatile fatty acid within a concentration range of not less than 9500 ppm and not more than 10500 ppm.

When measuring the concentration of volatile fatty acids, it may be desirable to have a high measurement accuracy of concentration around 10000 ppm. With the above method [17], since the calibration curve for volatile fatty acids is a straight line passing through the first point of the first sample specimen with a concentration of volatile fatty acids of not less than 9500 ppm and not more than 10500 ppm and the origin on the first coordinate system, the measurement accuracy of concentration of volatile fatty acids around 10000 ppm can be improved.

[18] In some embodiments, in any one of the above methods to [17], the component contained in the specimen includes ammonia nitrogen. The step of setting the calibration curve includes: a step (S165) of preparing a second sample specimen (70) containing the ammonia nitrogen of known concentration; a step (S166) of supplying the titrant to the second sample specimen; a step (S167) of plotting a second point (74) corresponding to concentration of the ammonia nitrogen contained in the second sample specimen on a second coordinate system (72) with a horizontal axis representing the amount of the titrant and a vertical axis representing the concentration of the ammonia nitrogen; and a step (S168) of defining a straight line that passes through the second point and an origin of the second coordinate system as the calibration curve (L2) of the ammonia nitrogen.

With the above method [18], it is possible to generate a calibration curve that improves the accuracy of measuring the concentration of volatile fatty acid in the specimen.

[19] In some embodiments, in the above method [18], the second sample specimen contains the ammonia nitrogen within a concentration range of not less than 4500 ppm and not more than 5500 ppm.

When measuring the concentration of ammonia nitrogen, it may be desirable to have a high measurement accuracy of concentration around 5000 ppm. With the above method [18], since the calibration curve for ammonia nitrogen is a straight line passing through the second point of the second sample specimen with a concentration of ammonia nitrogen of not less than 4500 ppm and not more than 5500 ppm and the origin on the second coordinate system, the measurement accuracy of concentration of ammonia nitrogen around 5000 ppm can be improved.

[20] In some embodiments, in the above method [18], the second sample specimen contains the ammonia nitrogen within a concentration range of not less than 3800 ppm and not more than 4200 ppm.

In measuring the concentration of ammonia nitrogen when the temperature of the specimen is medium temperature, it may be desirable to have a high measurement accuracy of concentration around 4000 ppm. With the above method [19], since the calibration curve for ammonia nitrogen is a straight line passing through the second point of the second sample specimen with a concentration of ammonia nitrogen of not less than 3800 ppm and not more than 4200 ppm and the origin on the second coordinate system, the measurement accuracy of concentration of ammonia nitrogen around 4000 ppm can be improved.

[21] In some embodiments, in the above method [18], the second sample specimen contains the ammonia nitrogen within a concentration range of not less than 2850 ppm and not more than 3150 ppm.

In measuring the concentration of ammonia nitrogen when the temperature of the specimen is high temperature, it may be desirable to have a high measurement accuracy of concentration around 3000 ppm. With the above method [20], since the calibration curve for ammonia nitrogen is a straight line passing through the second point of the second sample specimen with a concentration of ammonia nitrogen of not less than 2850 ppm and not more than 3150 ppm and the origin on the second coordinate system, the measurement accuracy of concentration of ammonia nitrogen around 3000 ppm can be improved.

### REFERENCE SIGNS LIST

1 Waste treatment system
2 Reforming device
4 Methane fermenter
6 Water quality measurement device
60 First sample specimen
62 First coordinate system
64 First point
70 Second sample specimen
72 Second coordinate system
74 Second point
1 Influence function
L1 First calibration curve
L2 Second calibration curve
S1 Target pH setting step
S2 pH range setting step
S4 Dilution step
S7 First sulfuric acid supply step
S8 Alkalinity calculation step
S9 Second sulfuric acid supply step
S10 Degassing step
S11 First sodium hydroxide aqueous solution supply step
S12 Volatile fatty acid concentration calculation step
S13 Second sodium hydroxide aqueous solution supply step
S14 Ammonia nitrogen concentration calculation step
S16 Calibration curve setting step
S161 First sample specimen preparation step
S162 First sample specimen supply step
S163 First plotting step
S164 First calibration curve generation step
S165 Second sample specimen preparation step
S166 Second sample specimen supply step
S167 Second plotting step
S168 Second calibration curve generation step
V2 Concentration calculation titrant volume
W Waste
X Reformed material
Y1 Contents of methane fermenter
Y2 Specimen
Y3 Titrant

1. A water quality measurement method for measuring acid consumption or alkali consumption of a specimen and concentration of a component contained in the specimen by titration, comprising:
a step of setting a target pH corresponding to the specimen;
a step of setting a pH range corresponding to a component contained in the specimen;
a step of supplying a titrant to the specimen after both the step of setting the target pH and the step of setting the pH range;
a step of calculating the acid consumption or alkali consumption of the specimen based on amount of the titrant supplied to the specimen until pH of the specimen reaches the target pH after starting to supply the titrant to the specimen; and
a step of calculating the concentration of the component contained in the specimen based on amount of the titrant supplied to the specimen until pH of the specimen changes from an upper limit to a lower limit of the pH range or from the lower limit to the upper limit after starting to supply the titrant to the specimen.

2. The water quality measurement method according to claim 1,
wherein the specimen is contents of a fermenter for microbially degrading a reformed material obtained by hydrolysis of waste.

3. The water quality measurement method according to claim 2,
wherein the fermenter is a methane fermenter which produces methane gas by microbially degrading the reformed material, and
wherein the step of setting the target pH includes setting the target pH based on an influence function that affects a production rate of the methane gas in the methane fermenter.

4. The water quality measurement method according to claim 3,
wherein the target pH is pH 7, and
wherein the step of calculating the acid consumption or alkali consumption of the specimen includes calculating the acid consumption of the specimen based on amount of the titrant supplied to the specimen until pH of the specimen reaches pH 7 after starting to supply the titrant to the specimen.

5. The water quality measurement method according to claim 2,
wherein the component contained in the specimen includes a volatile fatty acid, and
wherein the pH range includes a first pH range of not less than pH 2.8 and not more than pH 6.8.

6. The water quality measurement method according to claim 5,
wherein the first pH range is not less than pH 4.3 and not more than pH 5.3.

7. The water quality measurement method according to claim 5,
wherein the first pH range is corrected based on temperature of the specimen.

8. The water quality measurement method according to claim 2,
wherein the component contained in the specimen includes ammonia nitrogen, and
wherein the pH range includes a second pH range of not less than pH 7.8 and not more than pH 10.8.

9. The water quality measurement method according to claim 8,
wherein the second pH range is not less than pH 8.8 and not more than pH 9.8.

10. The water quality measurement method according to claim 8,
wherein the second pH range is corrected based on temperature of the specimen.

11. The water quality measurement method according to claim 1, further comprising a step of removing carbonic acid from the specimen after calculating the acid consumption of the specimen in the step of calculating the acid consumption or alkali consumption of the specimen,
wherein the step of calculating the concentration of the component contained in the specimen includes calculating the concentration of the component contained in the specimen based on amount of the titrant supplied to the specimen until pH of the specimen after removing carbonic acid changes from the lower limit to the upper limit of the pH range.

12. The water quality measurement method according to claim 1,
wherein the step of supplying the titrant to the specimen includes:
a first substep of supplying the titrant to the specimen at a first supply rate until pH of the specimen reaches the target pH after starting to supply the titrant to the specimen;
a second substep of supplying the titrant to the specimen at a second supply rate until pH of the specimen reaches the pH range after the first substep; and
a third substep of supplying the titrant to the specimen at a third supply rate until pH of the specimen reaches the upper limit or the lower limit of the pH range after the second substep, and
wherein the second supply rate is higher than each of the first supply rate and the third supply rate.

13. The water quality measurement method according to claim 1,
wherein the step of calculating the concentration of the component contained in the specimen includes calculating a concentration calculation titrant volume based on a pH change within the pH range of the specimen caused by the supply of the titrant, where the concentration calculation titrant volume is amount of the titrant supplied to the specimen until pH of the specimen changes from the upper limit to the lower limit of the pH range or from the lower limit to the upper limit.

14. The water quality measurement method according to claim 1, further comprising a step of diluting the specimen prior to the step of supplying the titrant to the specimen.

15. The water quality measurement method according to claim 1, further comprising a step of setting a calibration curve indicating a relationship between concentration of the component contained in the specimen and supply amount of the titrant prior to the step of supplying the titrant to the specimen,
wherein the step of calculating the concentration of the component contained in the specimen includes calculating the concentration of the component contained in the specimen based on the calibration curve and amount of the titrant supplied to the specimen until pH of the specimen changes from the upper limit to the lower limit of the pH range or from the lower limit to the upper limit after starting to supply the titrant to the specimen.

16. The water quality measurement method according to claim 15,
wherein the component contained in the specimen includes a volatile fatty acid,
wherein the step of setting the calibration curve includes:
a step of preparing a first sample specimen containing the volatile fatty acid of known concentration;
a step of supplying the titrant to the first sample specimen;
a step of plotting a first point corresponding to concentration of the volatile fatty acid contained in the first sample specimen on a first coordinate system with a horizontal axis representing the amount of the titrant and a vertical axis representing the concentration of the volatile fatty acid; and
a step of defining a straight line that passes through the first point and an origin of the first coordinate system as the calibration curve of the volatile fatty acid.

17. The water quality measurement method according to claim 16,
wherein the first sample specimen contains the volatile fatty acid within a concentration range of not less than 9500 ppm and not more than 10500 ppm.

18. The water quality measurement method according to claim 15,
wherein the component contained in the specimen includes ammonia nitrogen,
wherein the step of setting the calibration curve includes:
a step of preparing a second sample specimen containing the ammonia nitrogen of known concentration;
a step of supplying the titrant to the second sample specimen;
a step of plotting a second point corresponding to concentration of the ammonia nitrogen contained in the second sample specimen on a second coordinate system with a horizontal axis representing the amount of the titrant and a vertical axis representing the concentration of the ammonia nitrogen; and
a step of defining a straight line that passes through the second point and an origin of the second coordinate system as the calibration curve of the ammonia nitrogen.

19. The water quality measurement method according to claim 18,
wherein the second sample specimen contains the ammonia nitrogen within a concentration range of not less than 4500 ppm and not more than 5500 ppm.

20. The water quality measurement method according to claim 18,
wherein the second sample specimen contains the ammonia nitrogen within a concentration range of not less than 3800 ppm and not more than 4200 ppm.

21. (canceled)
